(19) United States
(12) Patent Application Publication
(10) Pub. No.: US 2010/0330192 A1
(43) Pub. Date: Dec. 30, 2010

(54) Method for the prevention of chronic inflammation associated diseases

(76) Inventor: Fabien De Meester, Marche-en Famenne (BE)

Correspondence Address: MCCRACKEN & FRANK LLP, 311 S. WACKER DRIVE, SUITE 2500, CHICAGO, IL, 60606, US

(21) Appl. No.: 12/449,686

(22) PCT Filed: Feb. 25, 2008
(86) PCT No.: PCT/EP2008/052258

§ 371 Date: Aug. 20, 2009

(30) Foreign Application Priority Data
Feb. 23, 2007 (EP) EP 07102973.0

(51) Int. Cl. A61K 35/12; A61K 35/20; A61K 35/34; A61K 35/54; A61K 36/00

(52) U.S. Cl. 424/522; 424/535; 424/548; 424/581; 424/725; 424/776

(57) ABSTRACT

The present invention is related to a method for the prevention and possibly the treatment of chronic diseases, preferably inflammatory associated chronic diseases that may affect an animal including a human, by the administration of a sufficient amount of a (functional) food or feed to the diet of this animal.

### FIELD OF THE INVENTION

The present invention is related to a method for the prevention and possibly the treatment of chronic diseases, preferably inflammatory associated chronic diseases that may affect an animal including a man (human), by the administration of a sufficient amount of a (functional) food or feed to the diet of this animal.

Inflammation is a complex biological response of vascular tissues to an harmful stimulus. Inflammations which run unchecked could lead to a host of diseases, especially acute or chronic diseases, such as hay fever, atherosclerosis and rheumatoid arthritis that are not down-regulated by the body.

In chronically inflamed tissues, a stimulus is persistent and therefore, a recruitment of monocytes is maintained. Existing macrophages are maintained in place and proliferation of these macrophages is stimulated.

Immune system is also often involved with inflammatory disorders demonstrated in both allergic reaction and in some myopathies. Furthermore, non-immune diseases with etiological origin in inflammatory process include cancer, atherosclerosis and ischemic heart (Ischemia) disease. Other disorders with inflammation include asthma, auto-immune diseases, chronic inflammation, chronic prostatitis, glomerulonephritis, hyper sensitivities, inflammatory bowel disease, pelvic inflammatory disease, reperfusion injury, rheumatoid arthritis, transplant rejection and vasculitis. An allergic reaction formerly known as type 1 hyper sensitivity is also the result of an inappropriate immune response triggering inflammation. Other hyper sensitivity reactions (type 2 and type 3) are mediated by antibody reactions and induces inflammation by attracting leukocytes, which damage surrounding tissue. Inflammatory myopathies are caused by the immune system inappropriately attacking component of muscle leading to site of muscle inflammation. They may occur in conjunction with other immune disorders such as systemic sclerosis and including dermatomyositis, polymyositis and inclusion body myositis. It is also known that high level of several inflammations relating markers such as IL-6, IL-8 and TNF-alpha are associated with obesity. During clinical study, inflammatory related molecule levels were reduced and increased levels of anti-inflammatory molecules were detected within 4 weeks after patient began a very low caloric diet. Furthermore, the association of systemic inflammation with insulin resistance and atherosclerosis has been also the subject of intensive research.

Prolonged inflammation, known as acute or chronic inflammation leads to a progressive shift of the type of cells which are present at the site of inflammation and is characterized by simultaneous destruction and healing of the tissue from the inflammatory process that could lead to chronic diseases, such as obesity, diabetes mellitus, cardio- and cerebrovascular diseases like hypertension or ischemia, auto-immune diseases (including diseases of inflammatory origin like arthritis or lupus), brain diseases (including neuro-degenerative diseases like Alzheimer disease, Parkinson disease, Huntington disease, multiple sclerosis, depression or schizophrenia), asthma, systemic sclerosis, allergies and cancer.

Therefore, in the present description, the applicant will use for the same effect, the words “chronic inflammation”, “chronic inflammation associated disease” or “inflammatory disorders” that constitute a large and related group of disorders which underlies a variety of human diseases.

Cholesterol in the animal kingdom has been submitted to an evolutionary selective process, yet it sits and functions in animal cells for hundreds of million years. In the 6-million-year-old hominid species, cholesterol accounts for 70 g per 70 kg body weight (0.1% w:w) and is distributed over all organs and tissues through blood circulation.

Cholesterol has been evolutionary selected as a unique blood and tissue active component in animals evolving in a wild environment. In such a land-based environment, body fats in herbivorous animals are characterized by a ˜1:1 ratio between the two plant essential fatty acids (EFAs), linoleic acid (LA, C18:2ω6) and alpha-linolenic acid (ALA, C18:3ω3).

It exists a need especially in the animal population for a reduction of these chronic diseases by a modification of the diet of animals preferably in mammals including the diet of humans.

These modifications should lead to a reduction of cholesterol-related chronic diseases for improving the health of animals, especially mammals subjects including human patients and for reducing the development of chronic diseases especially chronic inflammation and chronic inflammation associated diseases or disorders.

Verschuren et al. indicated that factor other than cholesterol, but diet-related are important for the prevention of heart disease (Serum total cholesterol and long-term coronary heart disease mortality in different cultures. Twenty-five-year follow-up of the seven countries study JAMA 1995; 274: 131-136). Moreover, the inventors previously demonstrated that the diets the study of Verschuren have very different ω6 & ω3 contents.

### SUMMARY OF THE INVENTION

The present invention is related to the use of (or to a method of prevention and possibly treatment) which comprises the step of adding to the diet of an animal) a sufficient amount of a (functional) natural (i.e. non genetically modified) food or feed composition inducing in total blood, serum, plasma or yolk of an animal, an HUFAs ω6 of about 25% (with a variance of about 5%) for the manufacture of a medicament to be administrated to this animal for an efficient prevention and possibly an efficient treatment of chronic diseases especially chronic inflammatory associated diseases affecting this animal, being preferably a mammal, including a human.

A functional and natural food or feed composition means a composition present in the diet of an animal, preferably a mammal, including a human, which is made of natural ingredients (non genetically modified ingredients obtained from non genetically modified plants or animals). Such restriction to natural compound is preferred, because national and European authorities do not accept a presence of genetically modified organisms or their portion in a composition destinated to domestic animals or humans. This percentage should be limited to 0.9% according to the European authorities.

Furthermore, it exists also an important discussion in human population regarding the drawbacks of genetically modified ingredients, especially genetically modified plants.

The food or feed composition according to the invention may comprise different ingredients of animal, vegetal or mineral (salt) origin.

Therefore, in the present invention, it is not possible to define the characteristic of the specific composition according to the invention, because it could be made of different ingredients having different ratios of (ω6 and ω3 polyunsaturated) fatty acids, but the mixture of these different ingredients will allow the preparation of a composition which is suitable for the diet of an animal, preferably a mammal, including a human, to induce in total blood, serum, plasma or yolk of the animal an HUFAs ω6% of about 25%, with a possible variance of 5%.

However, if the ingredients of such food or feed composition are of animal origin, they have preferably a ratio of ω6:ω3 polyunsaturated fatty acids PUFAs=1:1 with a variance of about 10%, preferably with a variance of about 5%. Examples of such ingredients of animal origin are egg, milk, meat, blood, skin, fat, fish, shell fish or a mixture thereof.

However, an ingredient comprising a higher concentration of ω3 polyunsaturated fatty acids may be added in order to obtain an efficient balance of ω6 and ω3 fatty acids in the composition.

The ingredients of the composition according to the invention could be also of a vegetable origin and may have preferably a ratio of ω6:ω3 essential fatty acids EFAs=1:3 with a variance of about 10%, preferably with a variance of about 5%.

Advantageously, these ingredients of vegetable origin are preferably selected from the group consisting of oils, vegetables roots or seeds possibly present in various compositions of vegetable origin, such as bread, paste or cookies. The composition may further comprise also suitable amount of carbohydrates, amino-acids (or proteins), anti-oxidants, vitamins and minerals (salt).

According to the invention, the inflammatory associated chronic diseases (inflammatory chronic disease or a disease of inflammatory origin) affecting this animal, preferably a mammal, including a human, are selected from the group consisting of obesity, diabetes mellitus, cardio- and cerebro-vascular diseases (atherosclerosis, hypertension, Ischemia) auto-immune diseases (including diseases of inflammatory origin, such as lateral Amyotrophic sclerosis, arthritis or lupus), brain diseases (including neurodegenerative diseases, such as Alzheimer disease, Parkinson disease, Huntington's disease, multiple sclerosis, depression or schizophrenia), asthma, systemic sclerosis, allergies and cancer.

Another aspect of the present invention is related to a method for the preparation of the functional and natural food or feed composition according to the invention which comprises ingredients of animal and/or vegetable origin. This method comprises the step of
identifying the percentage of fatty acids in each usual ingredient present in the recipe of the composition, and
modifying the recipe of this composition by mixing one or more of these usual ingredients with one or more additional ingredient(s) of animal or vegetable origin to obtain a composition inducing HUFAs ω6 of about 25% (with a variance of about 5%) in total blood, serum, plasma or yolk of an animal preferably by comprising a composition of animal HUFAs ω6 of 25% (with a variance of about 10% or 5%) and of vegetable EFAs ω6 of 25% (with a variance of about 10% or 5%).

### DETAILED DESCRIPTION OF THE INVENTION

Essential fatty acids (EFAs; linoleic acid, LA, C18:2ω6 and α-linolenic acid ALA, C18:3 ω3) are from vegetable or animal origin, while highly unsaturated fatty acids (HUFAs) are derived by an animal from EFAs (PUFA is the sum of EFA and HUFA).

The inventors have identified unexpectedly that an advantageous 25% ω6 in blood total, serum, plasma or yolk HUFAs (%ω6 HUFAs=25) is equivalent to the 1:1 ratio in serum PUFAs (ω6:ω3-PUFAs=1:1) in an animal, preferably a mammal, including an human and that this equivalence depends neither upon the type of diet (vegans, vegetarians, omnivorous, fish- or meat-based), neither upon the species (human, others mammals or birds), nor upon the latitude on earth where they live and dwell (Poles, Temperate Zones, Tropical and Sub-Tropical Zones, Equator, East, West, Continental).

Therefore, it seems that there is an universal rule among biological species on earth that define this percentage of ω6-HUFAs=25 and/or ω6:ω3-PUFAs=1:1 as an ideal blood and/or serum/yolk environment for moderate, cause-effect proportionate, healthy tissue-inflammatory responses to take place. Deviating from that gene-compliant standard may lead to chronic inflammation and associated diseases on the long run.

The inventors have identified that the proportion of ω6 in blood total highly unsaturated fatty acids (% ω6-HUFAs) is an accurate index of these tissues pro-inflammatory state, on the one hand, of dietary intake of polyunsaturated fatty acids (PUFAs), on the other hand and to the potentialization of the harmful effect of blood cholesterol. 25% ω6 in blood total HUFAs appears as an ideal diet-derived safeguard against these tissues inflammations and development of the mentioned chronic diseases.

Within diet comprising essential fatty acids, ratios are important, not amounts. Essential fatty acids (EFAs) are linoleic acid (LA C18:2 ω6) and alpha-linolenic acid (ALA, C18:3 ω3). It is now known that no more than 1% of daily intake energy (DEI) is needed as LA. Provided that threshold amount is reached, then a 1:3 ratio of LA:ALA is all what it takes to reach 25% ω6-HUFAs in total blood lipids and, as far as this dietary ω6:ω3-EFAs=1:3 ratio is maintained, an increase in the daily intake of LA will have no effect on the blood proportion of ω6 in total HUFAs. As there appears to be no absolute requirement for ω3-HUFAs (and therefore game and fish) in the human's diet, a vegan or a vegetarian diet can be perfectly fine with regards to human needs.

However, one must realise that today's modern food environment, loaded with hidden omega-6 fatty acids (LA,AA) does require the omnipresence of compensating ω3-HUFAs. This notion of ratios rather than the amount is of importance from a geographic perspective. Human populations live under different latitudes where sunshine and fats are differently distributed. For instance, cold polar environments favour HUFAs and EFAs, rainy temperatured latitudes favour EFAs and MUFAs (mono unsaturated fatty acids), and sunny tropical and equatorial latitudes favour MUFAs and SAFAs (saturated fatty acids). In wild-type environments under these different latitudes, ancient diets provide PUFAs that favour this preferred 25% ω6-HUFAs in total blood lipid and/or the ω6:ω3-PUFAs=1:1 in total serum lipids. However, ω6-rich grains and grain-fed livestock, thus not cholesterol and saturated fats, appear as the most single identified health care concern in modern man's diet around the world, including that of modern Inuits, Japanese, and Mediterranean. Moreover, those who were the least exposed to dietary PUFAs (Mediterranean zones and below) are also those who are at the highest risk of developing chronic diseases (and also obesity) when fed with ω6 rich modern foods, since this regimen is comparatively poor in ω3, needed to restore the ideal ratio.

Furthermore, the inventors have discovered that when applied to modern livestock (poultry), a balanced ratio of essential fatty acids (ω6:ω3 EFAs or PUFAs=1:1) in the animal (bird) body fat translates unexpectedly into a ˜1:3 ratio of ω6 to ω3 in its blood (and yolk of the obtained egg) highly unsaturated fatty acids (ω6:ω3 HUFAs=1:3).

It appears that Nature has selected cholesterol as an ideal tissue active component for land-based herbivorous animals complying with the following rules in terms of body fat and blood (yolk) fatty acid distribution (see table 1). Concurrently, such body fat and blood (yolk) fatty acid composition, when associated with the right diet-derived balance of essential amino acids, antioxidant vitamins and minerals, must be ideal for circulating lipoproteins (blood cholesterol) and tissue health & homeostasis, in land-based herbivorous animal species.

### CHD-Mortality and HUFAs in Man: Epidemiological Studies

Human prospective epidemiological evidence tends to show that the proportion of ω6 in blood total HUFAs is a potentially accurate parameter to estimate the risk of developing chronic diseases (Non-communicable diseases or NCDs), including obesity, diabetes mellitus, cardiovascular disease (CVD), hypertension, stroke, and some type of cancers, that are multigenic and multifactorial), such as exemplified by WEM Lands for coronary heart disease (CHD), and that the preferred proportion of 25% ω6 in blood total HUFAs (ω6:ω3 HUFAs=1:3) is an ideal epidemiologically-derived diet-related make-up in terms of protection against onset and development of such chronic diseases.

It is remarkable that the epidemiologically-determined safest blood HUFAs composition in man (25% ω6 HUFAs or ω6:ω3 HUFAs=1:3) is consistent with that naturally established in wild land-based herbivorous animals, and that the latter naturally establishes itself in modern livestock raised on a wild-type plan diet.

### From Nutrition Facts to Nurturing Facts

Epidemiological evidence tends to support the view that Classic Nutrition Facts could be advantageously substituted for Modern Nurturing Facts to show essential rather than the non-essential fat content of the food (see table 2). Saturated and mono-unsaturated fats as well as cholesterol are non-essential for man. Saturated fats and cholesterol are secondary risk factors for chronic human diseases, whereas essential and highly unsaturated fatty acids determine body tissue composition, health and homeostasis.

The contribution of balanced Egg such as described in the European Patent EP1282367B1 to blood total ω6 HUFAs is calculated from an empirical equation derived by WEM Lands that is available on the US NIH website: http://efaeducation.nih.gov/sig/dietbalance.html and/or at http://www.sbsoft.be/columbus-concept-5.html

The mathematical model can be used to accurately test a diet or to approximately test a specific food item for its potential contribution to HUFA-related tissue inflammatory status and, in turn, to evaluate the potential risk of developing a chronic disease by keeping on such a diet for a long time or by eating such food item on a regular basis (Table 3).

This interactive learning website can also be used to help determine a potentially ideal dietary essential fatty acid distribution that is needed to maintain tissue health and homeostasis. In particular, linoleic acid (LA, C18:2 ω6) must be taken into consideration here since saturation of fatty acid physiological pathways leading to endogenous synthesis of eicosanoids (autacoids) in rat and man appears to require less than 1.0% of D.E.I. (22.22 Cal or 2.5 gm) as LA. Intakes in excess of that threshold seem to lead to impairment of ω3-HUFAs synthesis and accretion in tissues and organs. Computational analysis of US NIH website equation leads to an optimized dietary ratio of plant fats, ie ω6:ω3-EFAs=1:3 (25% ω6-EFA/75% ω3-EFA) which maintains a proportion of 25% ω6 in blood total HUFAs. Interestingly, this distribution of essential fats is typical of ubiquitous greens and ancient seeds, such as flax, chia and perilla, on which man and wild-land based herbivorous animals have evolved.

ω6-rich grain-extracted table oils were not part of the food chain until Modern Agriculture came into play. From the classification obtained in table 3, it appears that most table oils seem to contribute to tissue inflammation and the onset of chronic diseases, so they could not have supported man's inception. Only certain Olive Oil (low in linoleic acid), Columbus Oil (described in the Patent Application WO2005/020698 and which is a corrected olive oil), chia, flax and perilla oils tends to a composition that is compatible with man's tissue homeostasis. Modern meat obtained from livestock fed ω6-rich grains is also inflammatory, because of its high content of animal-derived ω6-HUFAs.

Among suitable functional food or feed composition to be used in the present invention, the person skilled in the art may select ocean fatty fishes or food or feed composition comprising them that are balancing foods in a modern type diet. Thanks to their high content in ω3-HUFAs, they can help in reducing the pro-inflammatory properties of modern oils & fats, and meats.

Very important is the fact that dairy products—including full fat milk, butter and cream—are low in essential fatty acids and do not contribute to HUFA-related tissue inflammation, yet they may contribute to increases in the classic TC:HDL and LDL:HDL ratios because of their saturated fatty acids content. However, the person skilled in the art may also obtain wild-type milk that present substantially lowered ratios of medium to long chain fatty acids (MCFA: −25%; LCFA: +25%). Therefore, wild-type dairies all belong to a healthy balanced diet where total fat and energy are kept under control.

Last but not least, the person skilled in the art should also take into consideration published and current recommendations for the use of fatty acid composition ((a) Simopoulos A P et al., Essentially of and recommended dietary intakes for ω-6 and ω-3 fatty acids. Food Rev International 2000; 16:113-117; (b) Scientific Committee on Food. Report on the Revision of Essential Requirements of Infant Formulae and Follow-on Formulae. SCF/CS/NUT/IF/65 Final (18 May 2003), EU Commission, Health & Consumer Protection Directorate General).

Genetically speaking, human beings have not changed over the 10,000 years since the development of modern agriculture. Healthy human beings exhibit balanced body fats (ω6:ω3-EFAs=1:1) and/or a low proportion of ω6 in blood total HUFAs (ω6 in HUFAs=25%). These compositions can be obtained and maintained through selection of land-based wild-type diets, complying with the same ratios when from animal origin (game, river fish) and in favour of omega-3 fatty acids (ω6:ω3-EFAs=1:3) when from plant origin (leafy vegetables, some seeds & nuts).

TABLE 1

| Fatty acid (%) | Hen's body fat | Yolk total lipids |
|---|---|---|
| C16:0 | 12.90 | 19.34 |
| C18:0 | 5.43 | 9.18 |
| C16:1ω7 | 2.34 | 3.17 |
| C18:1ω9 | 33.92 | 37.74 |
| C18:2ω6 | 21.16 | 13.59 |
| C18:3ω3 | 21.16 | 11.69 |
| C20:4ω6 | 0.04 | 0.81 |
| C20:5ω3 | 0.03 | 0.28 |
| C22:5ω3 | 0.02 | 0.43 |
| C22:6ω3 | 0.04 | 1.86 |
| ω6:ω3 EFAs | ~1:1 | — |
| ω6:ω3 HUFAs | — | ~1:3 |
| ω6:ω3 PUFAs | ~1:1 | ~1:1 |

TABLE 2

| Classic Nutrition Facts | CE | | Modern Nurturing Facts | | CE |
|---|---|---|---|---|---|
| Energy (C/KJ) | 75 | | Energy (C/KJ) | | 75 |
| Saturated fat (g) | 1.5 | | D.E.I. (%) | | 3.75 |
| Monounsaturated fat (g) | 2.0 | | Essential fat (mg) | LA | 620 |
| Polyunsaturated fat (g) | 1.5 | [symbol] | | ALA | 550 |
| Cholesterol (mg) | 210 | | Highly unsaturated | ω6 HUFA | 40 |

| | | | |
|---|---|---|---|
| Vitamins (RDI) & Minerals (RDI) | fat (mg) | ω3 HUFA | 110 |

| | |
|---|---|
| Contribution of 1 Columbus Egg (CE) | 23% |
| to blood total ω6 HUFAs | |

Vitamins (RDI) & Minerals (RDI)
CE: Columbus Egg (One 65-g egg); D.E.I.: daily energy intake, RDI: recommended daily intake

TABLE 3
Traditional foods as per their predicted potential contribution
to tissue inflammation and development of chronic diseases

| | | | | | Blood | |
|---|---|---|---|---|---|---|
| | | | % | % | % | |
| | % | % | ω6 | ω3 | ω6 | Predicted |
| Oils & fats | LA | ALA | HUFAs | HUFAs | HUFAs | Risk |
| Sunflower oil | 61 | 0.1 | — | — | 76 | +++ |
| Grapeseed oil | 68 | 0.5 | — | — | 76 | +++ |
| Corn oil | 51 | 1.0 | — | — | 74 | +++ |
| Peanut oil | 35 | 0.1 | — | — | 73 | +++ |
| Wheat germ oil | 55 | 7 | — | — | 70 | +++ |
| Soybean oil | 54 | 7.5 | — | — | 70 | +++ |
| FAO/WHO 1994 | 11.1 | 0.92 | 0.74 | 0.37 | 70 | +++ |
| Walnut oil | 62 | 12 | — | — | 68 | +++ |
| Olive oil (1) | 13 | 0.6 | — | — | 63 | ++ |
| Palm oil | 9 | 0.25 | — | — | 58 | ++ |
| Canola oil | 20 | 10 | — | — | 54 | ++ |
| Std egg | 17.03 | 0.66 | 2.03 | 1.17 | 50 | + |
| Std red meat | 22.60 | 2.66 | 3.70 | 1.21 | 49 | + |
| Std white meat | 20.64 | 2.20 | 5.83 | 2.26 | 46 | + |
| Olive oil (2) | 5 | 0.6 | — | — | 47 | + |
| Columbus oil | 7 | 7 | — | — | 38 | 0 |
| Col red meat | 24.73 | 21.36 | 1.96 | 3.04 | 32 | 0 |
| Col white meat | 22.02 | 15.07 | 4.40 | 6.68 | 30 | 0 |
| Columbus egg | 13.59 | 11.69 | 0.81 | 2.57 | 27 | 0 |
| Coconut oil | 1.5 | 0.1 | — | — | 25 | 0 |
| Chia oil | 19 | 64 | — | — | 24 | 0 |
| Flax oil | 15 | 57.5 | — | — | 22 | 0 |
| Greek egg | 6.10 | 2.63 | 2.67 | 4.10 | 22 | 0 |
| Salmon | 5.20 | 5.30 | 9.80 | 28.80 | 20 | 0 |
| Perilla oil | 12.6 | 63.2 | — | — | 18 | 0 |
| Trout | 5.50 | 6.00 | 4.30 | 21.20 | 12 | − |
| Full fat milk | 3.5 | 1.0 | — | — | 11 | − |
| Mackerel | 1.28 | 0.07 | 0.37 | 18.84 | 2 | −− |
| Atlantic herring | 0.78 | 0.04 | 0.27 | 7.20 | 2 | −− |
| Pacific herring | 0.67 | 0.07 | — | 8.11 | 1 | −− |

“Predicted HUFA-related Risk” (correlated here to the contribution of the food to % ω6 in blood total HUFAs) is calculated from US NIH website

http://efaeducation.nih.gov/sig/dietbalancel.html for an intake of 20 g edible oil, 100-g edible egg (8.4 g total fatty acids), meat & fish (4.75 g total fatty acids), and milk (3.8 g total fatty acids) when contributing to a 30% fat-containing diet (2,222 Cal). Fatty acid compositional data are from The Lipid Handbook (Gunstone F D, Harwood J L & Padley F B, 2^{nd} Ed), Chapman & Hall, 1994, ISBN 0 412 43320 6. Data on Chia oil are retrieved from www.eatchia.com Greek Ampelistra egg is from Simopoulos A P & Salem N Jr. N-3 fatty acids in eggs from range-fed Greek chickens. N Engl J Med 1989; 321:1412 (letter). FAO/WHO 1994 were recommended standards for infant formula; here computing was made for <1 yr of age infant fed 900 ml milk formula daily. “Col” stands here for Columbus.

1. Use of a sufficient amount of a natural functional food or feed composition inducing in total blood, serum, plasma or yolk of an animal an HUFAs ω6 of about 25% with a variance of about 5%, for the manufacture of a medicament for a prevention and possibly a treatment of a chronic disease affecting said animal.

2. The use according to claim 1, wherein the chronic disease is an inflammatory chronic disease or a disease of inflammatory origin.

3. The use according to claim 2, wherein the food or feed composition comprises ingredients of animals origin selected from the group consisting of milk, egg, meat, fat, shell fish, fish or a mixture thereof.

4. The use according to claim 3, wherein the food or feed composition comprises ingredients of vegetal origin selected from the group consisting of oil, vegetable roots or seeds or a mixture thereof.

5. The use according to claim 1, wherein the animal is a domestic animal, preferably poultry.

6. The use according to claim 1, wherein the animal is a mammal.

7. The use of claim 6, wherein the mammal is a human.

8. The use according to claim 1, wherein the food or feed composition further comprises carbohydrates essential amino-acids, proteins, anti-oxidants, vitamins and minerals.

9. A method for obtaining a functional and natural food or feed composition comprising ingredients, especially fatty acids of animal and/or vegetable origin and inducing in total blood, serum, plasma or yolk of an animal an HUFAs ω6 of about 25% with a variance of about 5%, which comprises the step of
identifying the percentage of fatty acids in each usual ingredient present in the recipe of the composition, and
modifying the recipe of the said composition by mixing one or more of the said usual ingredients with one or more additional ingredient(s), especially fatty acids of animal or vegetable origin to obtain a composition of animal HUFAs ω6 of about 25% with a variance of about 5% and of vegetable EFAs ω6 of about 25% with a variance of about 5%.

10. Method for a prevention and possibly a treatment of a chronic disease, the method comprising the step of adding to the diet of an animal a sufficient amount of a natural functional food or feed composition inducing in total blood, serum, plasma or yolk of an animal an HUFAs ω6 of about 25% with a variance of 5%.

11. The method according to claim 10, wherein the chronic disease is an inflammatory chronic disease or a disease of inflammatory origin.

12. The method according to claim 10, wherein the food or feed composition comprises ingredients of animals origin selected from the group consisting of milk, egg, meat, fat, shell fish, fish or a mixture thereof.

13. The method according to claim 10, wherein the food or feed composition comprises ingredients of vegetal origin selected from the group consisting of oil, vegetable roots or seeds or a mixture thereof.

14. The method according to claim 10, wherein the animal is a domestic animal, preferably poultry.

15. The method according to claim 10, wherein the animal is a mammal.

16. The method according to claim 15, wherein the animal is a human.

17. The method according to claim 10, wherein the food or feed composition further comprises carbohydrates essential amino-acids, proteins, anti-oxidants, vitamins and minerals.
